(19) United States
(12) Patent Application Publication
(10) Pub. No.: US 2025/0333672 A1
(43) Pub. Date: Oct. 30, 2025

(54) BAG

(71) Applicants: CANON MEDICAL SYSTEMS CORPORATION, Otawara-shi (JP); Canon Kabushiki Kaisha, Tokyo (JP)

(72) Inventors: Hirotoshi TAHARA, Ota (JP); Sotaro TAKI, Chofu (JP)

(73) Assignees: CANON MEDICAL SYSTEMS CORPORATION, Otawara-shi (JP); Canon Kabushiki Kaisha, Tokyo (JP)

(21) Appl. No.: 19/187,250

(22) Filed: Apr. 23, 2025

(30) Foreign Application Priority Data
Apr. 24, 2024 (JP) 2024-070356

(51) Int. Cl. C12M 1/00; C12M 3/00

(52) U.S. Cl. CPC C12M 23/14; C12M 23/40; C12M 23/48; C12M 29/00

(57) ABSTRACT

A bag according to an embodiment includes a housing part, a discharging part, and a discharging pipe device. The housing part includes a housing chamber that houses a liquid as a sample or a reagent. The discharging part includes a discharging path configured to discharge the liquid from the housing chamber. The discharging pipe device is connected to the discharging part, and discharges the liquid from the housing chamber via the discharging path. The discharging pipe device is connected to the discharging part to extend from the discharging part toward a direction having a horizontal direction component.

### CROSS-REFERENCE TO RELATED APPLICATIONS

This application is based upon and claims the benefit of priority from Japanese Patent Application No. 2024-070356, filed on Apr. 24, 2024, the entire contents of which are incorporated herein by reference.

### FIELD

Embodiments described herein relate generally to a bag.

### BACKGROUND

Conventionally, in preparing induced pluripotent stem cells (hereinafter, referred to as iPS cells), for example, samples and reagents are supplied from a plurality of bags to a device configured to prepare cells.

The bags in which the samples or the reagents are housed may be constituted of a soft material, for example.

### BRIEF DESCRIPTION OF THE DRAWINGS

FIG. 1 is a front view schematically illustrating a bag according to an embodiment;

FIG. 2 is a bottom view schematically illustrating the bag according to the embodiment;

FIG. 3 is a front view schematically illustrating a main body part according to the embodiment;

FIG. 4 is a diagram schematically illustrating the main body part at a IV-IV cross section in FIG. 3; and

FIG. 5 is a front view schematically illustrating a bag according to a modification.

### DETAILED DESCRIPTION

The bag according to the embodiment includes a housing part, a discharging part, and a discharging pipe device. The housing part includes a housing chamber that houses a liquid as a sample or a reagent. The discharging part includes a discharging path configured to discharge the liquid from the housing chamber. The discharging pipe device is connected to the discharging part, and discharges the liquid from the housing chamber via the discharging path. The discharging pipe device is connected to the discharging part to extend from the discharging part toward a direction having a horizontal direction component.

The following describes the bag with reference to the drawings. In the following embodiment, portions denoted by the same reference numerals are assumed to perform the same operation, so that redundant description is appropriately omitted. Content described in one embodiment is similarly applied to a modification in principle.

The bag according to the embodiment is a bag that houses a liquid as a sample or a reagent configured to prepare iPS cells, for example. For example, a plurality of the bags are housed in a cartridge configured to prepare iPS cells, and the cartridge is installed in an iPS cell manufacturing device. In a case of preparing iPS cells, samples and reagents are supplied from a plurality of the bags to the cartridge. Each of the bags is connected to a closed-system flow channel housed in the cartridge. A liquid housed in each bag is introduced into the closed-system flow channel, and used configured to prepare iPS cells. At this point, driving force for introducing the liquid into the closed-system flow channel is required. For example, the iPS cell manufacturing device is connected to the cartridge and applies a negative pressure to the closed-system flow channel in the cartridge to supply the driving force. The cartridge configured to prepare iPS cells is an example of a unit separate from the bag.

The sample is blood of a human body, for example. The reagent is a reagent required configured to prepare iPS cells, for example. As the reagent required configured to prepare iPS cells, a known reagent can be applied, so that description thereof is omitted.

An amount of the sample and the reagent used configured to prepare iPS cells is, for example, several milliliters to several tens of milliliters. Thus, for example, a small amount of the sample or the reagent is housed in the bag. The liquid housed in the bag is not limited to the sample or the reagent configured to prepare iPS cells.

Hereinafter, a vertical direction is assumed to be an upper and lower direction, a thickness direction of the bag of directions (horizontal directions) orthogonal to the upper and lower direction is assumed to be a front and rear direction, and a direction orthogonal to the upper and lower direction and the front and rear direction is assumed to be a left and right direction.

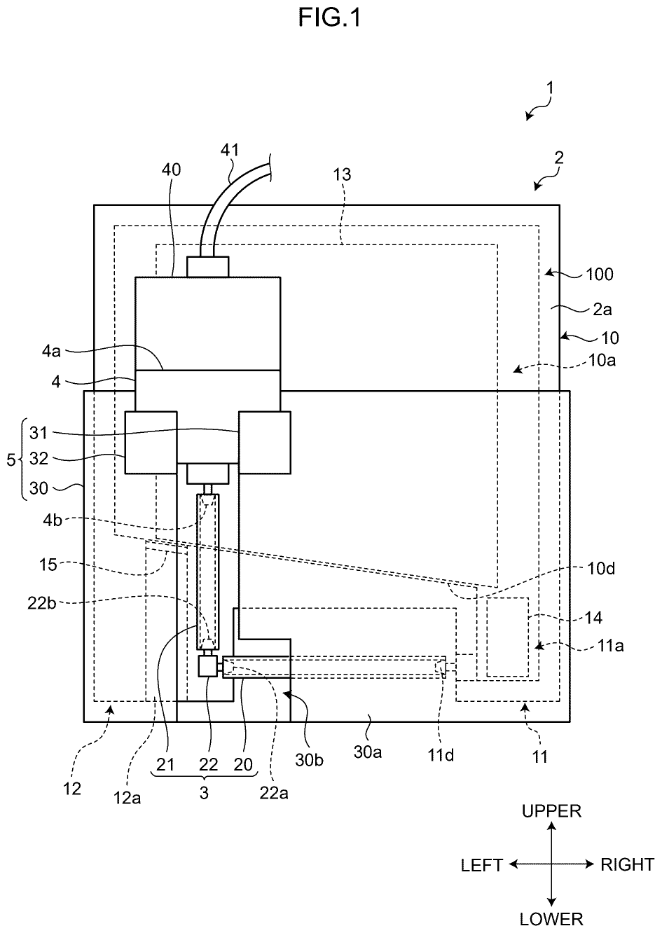

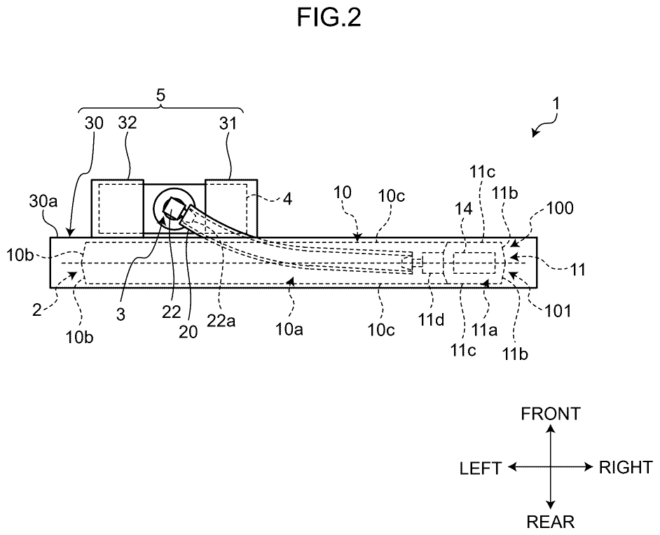

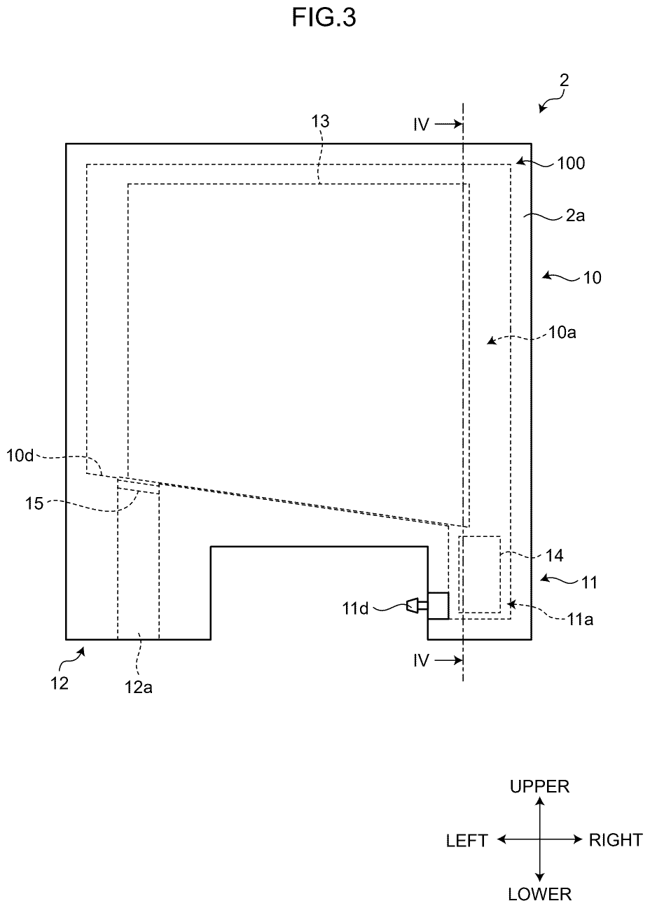

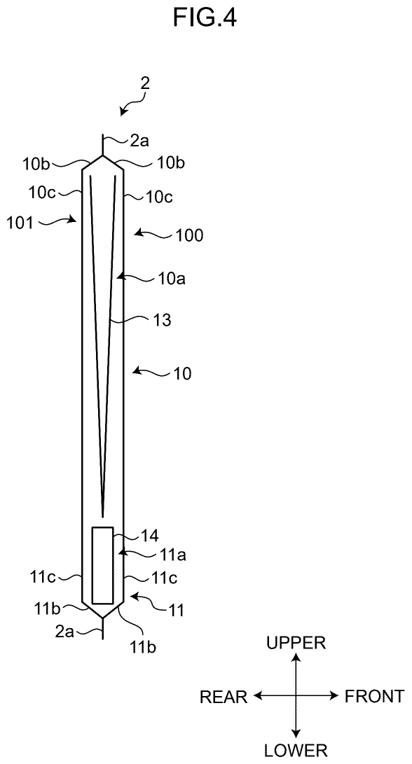

The following describes a bag 1 according to the embodiment with reference to FIG. 1 to FIG. 4. FIG. 1 is a front view schematically illustrating the bag 1 according to the embodiment. FIG. 2 is a bottom view schematically illustrating the bag 1 according to the embodiment. FIG. 3 is a front view schematically illustrating a main body part 2 according to the embodiment. FIG. 4 is a diagram schematically illustrating the main body part 2 at a IV-IV cross section in FIG. 3. In each of the drawings, some of hidden lines are indicated by dashed lines.

As illustrated in FIG. 1 and FIG. 2, the bag 1 includes the main body part 2, a discharging pipe device 3, a connector 4, and a holder 5.

As illustrated in FIG. 3, the main body part 2 includes a housing part 10, a discharging part 11, a filling part 12, an energizing part 13, a cylindrical member 14, and a sealing part 15.

As illustrated in FIG. 4, the housing part 10 is formed by joining part of a first sheet 100 with part of a second sheet 101. A peripheral part 2a is formed around the housing part 10. The peripheral part 2a is a portion where the first sheet 100 is joined with the second sheet 101. Each of the sheets 100 and 101 is constituted of a soft material. A material of each of the sheets 100 and 101 is set in accordance with a type of a liquid housed in the bag 1. The material of each of the sheets 100 and 101 is a material that does not react with a component of the liquid housed in the bag 1, and a material that does not adsorb a component of the liquid housed in the bag 1. The same applies to other members used for the bag 1.

The housing part 10 includes a housing chamber 10a. The housing chamber 10a houses a liquid as a sample or a reagent. The housing chamber 10a is formed of a peripheral wall part 10b and a side wall part 10c.

The peripheral wall part 10b is formed to rise from the peripheral part 2a. The peripheral wall part 10b formed on the first sheet 100 is formed to rise from the peripheral part 2a toward a front side. The peripheral wall part 10b formed on the second sheet 101 is formed to rise from the peripheral part 2a toward a rear side.

The side wall part 10c is formed to be connected to an end part of the peripheral wall part 10b on the opposite side of the peripheral part 2a. That is, the peripheral wall part 10b and the side wall part 10c formed on the first sheet 100 are formed to project toward the front side. The peripheral wall part 10b and the side wall part 10c formed on the second sheet 101 are formed to project toward the rear side.

In this way, the housing part 10 is formed to project toward an outer side. The outer side is opposite to sides of the first sheet 100 and the second sheet 101 on which the liquid is present in a state in which the liquid is housed in the housing chamber 10a.

A bottom part 10d of the housing chamber 10a is inclined toward a lower side as illustrated in FIG. 3. The bottom part 10d of the housing chamber 10a is inclined toward the discharging part 11. For example, the bottom part 10d of the housing chamber 10a is inclined so that it is positioned on the lower side as being closer to a right side.

The discharging part 11 is disposed on the lower side of the housing part 10. The discharging part 11 is disposed on an end part side of the housing part 10 in the left and right direction. For example, the discharging part 11 is disposed on a right end side of the housing part 10. The discharging part 11 is disposed to project toward the lower side from the housing part 10.

The discharging part 11 is formed by joining part of the first sheet 100 with part of the second sheet 101 (refer to FIG. 4). The housing part 10 and the discharging part 11 are integrally formed.

The discharging part 11 includes a discharging path 11a. The discharging path 11a communicates with a lower end of the housing chamber 10a. The discharging path 11a is formed to extend toward the lower side from the lower end of the housing chamber 10a. The discharging path 11a is a flow channel configured to discharge the liquid from the housing chamber 10a. Capacity of the discharging path 11a is smaller than capacity of the housing chamber 10a.

As illustrated in FIG. 4, the discharging path 11a is formed of a peripheral wall part 11b and a side wall part 11c.

The peripheral wall part 11b is formed to rise from the peripheral part 2a. The peripheral wall part 11b formed on the first sheet 100 is formed to rise from the peripheral part 2a toward the front side. The peripheral wall part 11b formed on the second sheet 101 is formed to rise from the peripheral part 2a toward the rear side.

The side wall part 11c is formed to be connected to an end part of the peripheral wall part 11b on the opposite side of the peripheral part 2a. That is, the peripheral wall part 11b and the side wall part 11c formed on the first sheet 100 are formed to project toward the front side. The peripheral wall part 11b and the side wall part 11c formed on the second sheet 101 are formed to project toward the rear side. In this way, the discharging part 11 is formed to project toward the outer side.

The discharging part 11 includes a connection joint 11d. As illustrated in FIG. 3, the connection joint 11d is disposed on a lower end side of the discharging part 11. The connection joint 11d is disposed at a left end of the discharging part 11. Part of the connection joint 11d is connected to the first sheet 100 and the second sheet 101. The connection joint 11d projects in a direction having a horizontal direction component. In other words, the connection joint 11d projects in a direction intersecting with the vertical direction. Specifically, part of the connection joint 11d projects toward a left side. The connection joint 11d may also be disposed to be inclined in the upper and lower direction with respect to the horizontal direction. A flow channel is formed in the connection joint 11d. The flow channel communicates with the discharging path 11a.

The filling part 12 is formed to extend toward the lower side from the housing part 10. The filling part 12 is disposed on an end part side in the left and right direction. For example, the filling part 12 is disposed on a left end side of the housing part 10. That is, the filling part 12 is disposed on the opposite side of the discharging part 11 in the left and right direction. For example, a lower end of the filling part 12 is disposed on a virtual line extended from the lower end of the discharging part 11 in the left and right direction.

The filling part 12 is formed by joining part of the first sheet 100 with part of the second sheet 101 (refer to That is, the housing part 10 and the filling part FIG. 4). 12 are integrally formed. The filling part 12 is formed so that the liquid fills the housing chamber 10a through the bottom part 10d of the housing chamber 10a. The filling part 12 includes a filling path 12a. The filling path 12a is a flow channel configured to fill the housing chamber 10a with the liquid. The filling path 12a is formed to extend in the upper and lower direction, for example.

The energizing part 13 is housed in the housing chamber 10a. The energizing part 13 is a leaf spring, for example. Specifically, as illustrated in FIG. 4, the energizing part 13 is formed to have a substantially V-shape in a lateral view. In a case in which the liquid is discharged from the housing chamber 10a, the energizing part 13 energizes the side wall part 10c of the housing part 10 forming the housing chamber 10a toward the outer side. Specifically, in a case in which the liquid is discharged from the housing chamber 10a, the energizing part 13 energizes the side wall part 10c of the housing part 10 forming the housing chamber 10a toward the front and rear direction. Repulsive force of the energizing part 13 is set so that the energizing part 13 is closed when the liquid in the housing chamber 10a is discharged and a pressure in the housing chamber 10a becomes smaller than atmospheric pressure.

The cylindrical member 14 is housed in the discharging path 11a. The cylindrical member 14 is formed in a cylindrical shape, for example. The cylindrical member 14 is formed to extend in the upper and lower direction, for example. The cylindrical member 14 is disposed so that the discharging path 11a is not blocked when the liquid is discharged from the housing chamber 10a. For example, the cylindrical member 14 is disposed not to be collapsed in the front and rear direction even in a case in which the liquid is discharged from the housing chamber 10a and the pressure in the discharging path 11a becomes smaller than atmospheric pressure.

As illustrated in FIG. 3, the sealing part 15 is disposed on an upper end side of the filling path 12a. The sealing part 15 is formed when the housing chamber 10a is filled with the liquid and the filling path 12a is blocked thereafter. The sealing part 15 is formed by joining the first sheet 100 with the second sheet 101, for example. The sealing part 15 interrupts the communication between the filling path 12a and the housing chamber 10a after the housing chamber 10a is filled with the liquid. The sealing part 15 is formed along the bottom part 10d of the housing chamber 10a. The sealing part 15 is preferably formed so that an upper end of the sealing part 15 is disposed on the same straight line as the bottom part 10d of the housing chamber 10a.

As illustrated in FIG. 1, the discharging pipe device 3 connects the discharging part 11 with the connector 4. The discharging pipe device 3 discharges the liquid from the housing chamber 10a via the discharging path 11a. The discharging pipe device 3 is connected to the discharging part 11 to extend from the discharging part 11 toward a direction having a horizontal direction component. The discharging pipe device 3 is disposed to extend from the discharging part 11 toward a direction having the horizontal direction component, and extend upward toward the connector 4. That is, the discharging pipe device 3 is formed in a substantially L-shape in a front view.

The discharging pipe device 3 includes a first tube 20, a second tube 21, and a joint 22. The first tube 20 is connected to the discharging part 11. Specifically, one end part of the first tube 20 is connected to the connection joint 11d of the discharging part 11. The first tube 20 is disposed to extend from the connection joint 11d toward a direction having a horizontal direction component. For example, the first tube 20 is disposed to extend from the connection joint 11d toward the left side. The first tube 20 may also be disposed to be inclined in the upper and lower direction with respect to the horizontal direction. The other end part of the first tube 20 is connected to a first connection joint 22a of the joint 22.

The joint 22 connects the first tube 20 with the second tube 21. The joint 22 is formed in a substantially L-shape. A flow channel is formed in the joint 22. The joint 22 changes a flowing direction of the liquid flowed in from the first tube 20 to an upper side. The joint 22 includes the first connection joint 22a and a second connection joint 22b.

The first tube 20 is connected to the first connection joint 22a. The second tube 21 is connected to the second connection joint 22b.

The second tube 21 is connected to the connector 4. One end part of the second tube 21 is connected to a lower surface of the connector 4. Specifically, one end part of the second tube 21 is connected to a connection joint 4b disposed on the lower surface of the connector 4. The second tube 21 is disposed to extend from the lower surface of the connector 4 toward the lower side. For example, the second tube 21 is disposed to extend from the lower surface of the connector 4 in the vertical direction. The second tube 21 may be disposed to be inclined from the lower surface of the connector 4 with respect to the vertical direction. The other end part of the second tube 21 is connected to the second connection joint 22b of the joint 22.

The connector 4 connects the discharging pipe device 3 with an external connector 40. The connector 4 allows the liquid to flow into the external connector 40 from the discharging pipe device 3. The connector 4 is an aseptic connector. The external connector 40 is a connector connected to a closed-system tube 41 that supplies the liquid to the cartridge configured to prepare iPS cells as a separate unit. The external connector 40 is an aseptic connector.

The holder 5 holds the main body part 2 and the connector 4. That is, the holder 5 holds the housing part 10, the discharging part 11, and the connector 4. The holder 5 includes a first holding part 30 and second holding parts 31 and 32. The first holding part 30 holds the main body part 2. An upper end of the first holding part 30 is opened. Part of the main body part 2 and part of the discharging pipe device 3 are inserted into the first holding part 30. Specifically, part of the housing part 10, the discharging part 11, and the filling part 12 are inserted into the first holding part 30. Additionally, part of the first tube 20 is inserted into the first holding part 30.

A notch part 30b is formed on a side wall part 30a of the first holding part 30. The side wall part 30a on which the notch part 30b is formed is, for example, a side wall part on the front side. Part of the notch part 30b is formed to extend in the upper and lower direction. The notch part 30b is formed in a substantially L-shape in a front view, for example. The notch part 30b may also be formed in a linear shape to extend in the upper and lower direction in a front view. The first tube 20 is inserted through the notch part 30b. That is, the first tube 20 is brought out from the first holding part 30 toward the front side via the notch part 30b.

The second holding parts 31 and 32 hold the connector 4. The second holding parts 31 and 32 are formed to project from the side wall part 10c of the first holding part 30 toward the front side. The second holding parts 31 and 32 are disposed on the upper side of the discharging part 11. The second holding parts 31 and 32 hold the connector 4 on the upper side of the discharging part 11. The second holding parts 31 and 32 are disposed to be separated in the left and right direction. An upper end and a left end of the second holding part 31 are opened. An upper end and a right end of the second holding part 32 are opened. Part of the connector 4 is inserted into the second holding parts 31 and 32. For example, the second holding parts 31 and 32 are formed so that a connecting surface 4a (upper end surface) of the connector 4 for the external connector 40 is arranged in the horizontal direction.

In the bag 1, after the housing chamber 10a is filled with the liquid via the filling part 12, the sealing part 15 is formed. The main body part 2 filled with the liquid and the discharging pipe device 3 are inserted into the first holding part 30, and held by the first holding part 30. The connector 4 is also inserted into the second holding parts 31 and 32, and held by the second holding parts 31 and 32. Due to this, the main body part 2 and the connector 4 are held by the holder 5.

The connector 4 is connected to the external connector 40 in a state of being held by the holder 5. For example, a user grasps the holder 5, and connects the connector 4 with the external connector 40.

When the liquid is supplied to the cartridge configured to prepare iPS cells, the inside of the closed-system tube 41 is caused to have a negative pressure. Due to this, the liquid flows into the closed-system tube 41 from the housing chamber 10a through the discharging part 11, the discharging pipe device 3, the connector 4, and the external connector 40. That is, the liquid in the housing chamber 10a is sucked, and flows into the closed-system tube 41. Specifically, after the liquid that has flowed into the discharging pipe device 3 from the housing chamber 10a via the discharging path 11a flows toward the left side through the discharging pipe device 3, the flowing direction thereof is changed to the upper side by the joint 22, and the liquid passes through the connector 4 and the external connector 40 to flow into the closed-system tube 41. The liquid is then supplied to the cartridge configured to prepare iPS cells.

When the liquid is discharged from the housing chamber 10a, the liquid housed in the housing chamber 10a flows into the discharging path 11a along the bottom part 10d of the housing chamber 10a. That is, when the liquid is discharged from the housing chamber 10a, the liquid housed in the housing chamber 10a is guided to the discharging path 11a along the bottom part 10d of the housing chamber 10a.

When the liquid is discharged from the housing chamber 10a, the liquid in the housing chamber 10a is sucked, so that the pressure in the housing chamber 10a becomes smaller than atmospheric pressure. Thus, the housing part 10 is pressed from the front and rear direction by atmospheric pressure, and the capacity of the housing chamber 10a is reduced. The housing part 10 is formed to project in the front and rear direction. Due to this, when the housing chamber 10a is pressed from the front and rear direction by atmospheric pressure and the capacity of the housing chamber 10a is reduced, the housing part 10 can be prevented from being wrinkled, and prevented from being narrowed.

The energizing part 13 is housed in the housing chamber 10a, so that, when the housing part 10 is pressed from the front and rear direction by atmospheric pressure, the side wall part 10c is energized toward the outer side by the energizing part 13. Due to this, when the housing chamber 10a is pressed from the front and rear direction by atmospheric pressure and the capacity of the housing chamber 10a is reduced, the housing part 10 can be prevented from being wrinkled, and prevented from being narrowed.

The discharging part 11 is formed to project in the front and rear direction. Due to this, when being pressed from the front and rear direction by atmospheric pressure, the discharging part 11 can be prevented from being wrinkled, and prevented from being narrowed.

The cylindrical member 14 is housed in the discharging path 11a, so that, when the discharging part 11 is pressed from the front and rear direction by atmospheric pressure, the discharging part 11 can be prevented from being narrowed.

In a bag according to a comparative example without using the present embodiment, for example, a discharging pipe is disposed to extend from the housing part toward the lower side. Thus, in the bag according to the comparative example, after the discharging pipe extends toward the lower side, for example, the discharging pipe is bent to be laid to the upper side. Thus, in the bag according to a comparative example, the discharging pipe is extended to the lower side, so that a dead volume is generated on the lower side.

The bag 1 according to the embodiment includes the housing part 10, the discharging part 11, and the discharging pipe device 3. The housing part 10 includes the housing chamber 10a that houses the liquid as a sample or a reagent. The discharging part 11 includes the discharging path 11a configured to discharge the liquid from the housing chamber 10a. The discharging pipe device 3 is connected to the discharging part 11, and discharges the liquid from the housing chamber 10a via the discharging path 11a. The discharging pipe device 3 is connected to the discharging part 11 to extend from the discharging part 11 toward a direction having a horizontal direction component.

Due to this, the bag 1 can prevent the discharging pipe device 3 from hanging down, and prevent a dead volume from being generated on the lower side of the housing part 10. For example, in a case in which a plurality of the bags 1 are arranged side by side to be used, the bags 1 can prevent a dead volume from being generated on the lower side of the arranged bags 1.

The discharging part 11 is disposed on the lower side of the housing part 10. The discharging path 11a communicates with the lower end of the housing chamber 10a. The bottom part 10d of the housing chamber 10a is inclined toward the discharging path 11a. That is, the bottom part 10d of the housing chamber 10a is inclined toward the lower side, and when the liquid is discharged from the housing chamber 10a, the liquid is guided to the discharging path 11a along the bottom part 10d of the housing chamber 10a.

Due to this, when the liquid is discharged from the housing chamber 10a, the liquid flows into the discharging path 11a communicating with the lower end of the housing chamber 10a along the bottom part 10d of the housing chamber 10a. Thus, in a case of discharging the liquid from the housing chamber 10a, the bag 1 can easily discharge the liquid from the housing chamber 10a to the discharging path 11a. In a case of discharging the liquid from the housing chamber 10a, the bag 1 can prevent the liquid from remaining in the housing chamber 10a. Accordingly, the bag 1 can accurately supply the liquid housed in the housing chamber 10a to the cartridge configured to prepare iPS cells. In other words, the bag 1 can supply a prescribed amount of liquid to the cartridge configured to prepare iPS cells.

The housing part 10 is formed by joining part of the first sheet 100 with part of the second sheet 101. The housing part 10 includes the peripheral wall part 10b rising from the peripheral part 2a as a joining portion of the first sheet 100 and the second sheet 101.

Due to this, when the liquid housed in the housing chamber 10a is sucked and discharged from the housing chamber 10a, the bag 1 can prevent the housing part 10 from being wrinkled due to the peripheral wall part 10b, and prevent the housing part 10 from being narrowed. Thus, in a case of discharging the liquid from the housing chamber 10a, the bag 1 can prevent the liquid from remaining in the housing chamber 10a. Accordingly, the bag 1 can accurately supply the liquid housed in the housing chamber 10a to the cartridge configured to prepare iPS cells.

The bag 1 also includes the energizing part 13 housed in the housing chamber 10a. When the liquid is discharged from the housing chamber 10a, the energizing part 13 energizes the side wall part 10c of the housing part 10 toward the outer side.

Due to this, when the liquid housed in the housing chamber 10a is sucked and discharged from the housing chamber 10a, the bag 1 can prevent the housing part 10 from being wrinkled, and prevent the housing part 10 from being narrowed by energizing the side wall part 10c toward the outer side by the energizing part 13. Thus, in a case of discharging the liquid from the housing chamber 10a, the bag 1 can prevent the liquid from remaining in the housing chamber 10a. Accordingly, the bag 1 can accurately supply the liquid housed in the housing chamber 10a to the cartridge configured to prepare iPS cells.

The discharging part 11 is formed by joining part of the first sheet 100 with part of the second sheet 101. The discharging part 11 includes the peripheral wall part 11b rising from the peripheral part 2a as a joining portion of the first sheet 100 and the second sheet 101.

Due to this, when the liquid housed in the housing chamber 10a is sucked and the liquid is discharged from the discharging path 11a, the bag 1 can prevent the discharging path 11a from being collapsed by the peripheral wall part 11b, and prevent the discharging path 11a from being narrowed. Thus, in a case of discharging the liquid from the housing chamber 10a, the bag 1 can prevent the liquid from remaining in the housing chamber 10a. Accordingly, the bag 1 can accurately supply the liquid housed in the housing chamber 10a to the cartridge configured to prepare iPS cells.

The bag 1 also includes the cylindrical member 14 housed in the discharging path 11a.

Due to this, when the liquid housed in the housing chamber 10a is sucked and the liquid is discharged from the discharging path 11a, the bag 1 can prevent the discharging path 11a from being narrowed by the cylindrical member 14. Thus, in a case of discharging the liquid from the housing chamber 10a, the bag 1 can prevent the liquid from remaining in the housing chamber 10a. Accordingly, the bag 1 can accurately supply the liquid housed in the housing chamber 10a to the cartridge configured to prepare iPS cells.

The discharging part 11 is formed integrally with the housing part 10.

Due to this, the bag 1 can cause part of the main body part 2 to function as the discharging path 11a. Thus, the bag 1 can connect the discharging pipe device 3 to the main body part 2 so that the discharging pipe device 3 extends from the discharging part 11 that is formed integrally with the housing part 10 toward a direction including a horizontal direction component. That is, the bag 1 can connect the discharging pipe device 3 to the main body part 2 without extending the discharging pipe device 3 toward the lower side. Thus, the bag 1 can suppress a dead volume on the lower side of the housing part 10.

The bag 1 also includes the connector 4 and the holder 5. The connector 4 connects the discharging pipe device 3 with the external connector 40, and allows the liquid to flow into the external connector 40 from the discharging pipe device 3. The holder 5 holds the housing part 10, the discharging part 11, and the connector 4.

Due to this, the user can connect the connector 4 to the external connector 40 in a state in which the holder 5 holds the housing part 10, the discharging part 11, and the connector 4. Thus, the bag 1 can prevent the discharging pipe device 3 from being pulled or bent when the connector 4 is connected to the external connector 40. Thus, the bag 1 can prevent the discharging pipe device 3 from being broken. The user can connect the external connector 40 to the connector 4 held by the holder 5 in a state of grasping the holder 5, for example. Thus, the user can easily perform work of connecting the connector 4 with the external connector 40. The bag 1 can also prevent the housing part 10 and the discharging part 11 from being wrinkled by holding the housing part 10 and the discharging part 11 by the holder 5. The bag 1 can stand by itself using the holder 5. Thus, for example, in a case of arranging a plurality of the bags 1, the bags 1 can be stably arranged.

The holder 5 holds the connector 4 on the upper side of the discharging part 11.

Due to this, when the discharging pipe device 3 is connected to the discharging part 11 to extend from the discharging part 11 in a direction having a horizontal direction component, the bag 1 can prevent the discharging pipe device 3 from hanging down from the main body part 2. Thus, the bag 1 can suppress a dead volume on the lower side.

The discharging pipe device 3 includes the first tube 20, the second tube 21, and the joint 22. The first tube 20 is connected to the discharging part 11. The second tube 21 is connected to the lower surface of the connector 4. The joint 22 connects the first tube 20 with the second tube 21.

Due to this, in the bag 1, the first tube 20 can be arranged to extend from the discharging part 11 along a direction having a horizontal direction component. In the bag 1, the second tube 21 can be arranged to extend along the vertical direction from the lower surface of the connector 4. That is, in the bag 1, the first tube 20 and the second tube 21 can be connected to the joint 22 while reducing bending of the first tube 20 and the second tube 21. Thus, the bag 1 can prevent the first tube 20 and the second tube 21 for connecting the discharging part 11 with the connector 4 from being broken. The bag 1 can also prevent the discharging pipe device 3 from hanging down from the main body part 2. Thus, the bag 1 can suppress a dead volume on the lower side.

The bag 1 also includes the filling part 12. The filling part 12 includes the filling path 12a configured to fill the housing chamber 10a with the liquid, and is formed to extend from the housing part 10 toward the lower side.

Due to this, the bag 1 can prevent lengths in the front and rear direction and the left and right direction from being increased. The filling part 12 is formed to extend toward the lower side that is the same direction as the direction in which the discharging part 11 is disposed with respect to the housing part 10. Thus, the bag 1 can effectively utilize a region on the lower side of the housing part 10. In the bag 1, the housing part 10 housed in the holder 5 can be supported from the lower side by the discharging part 11 and the filling part 12. Thus, the bag 1 can improve stability of the main body part 2 inserted into the holder 5.

The bag 1 also includes the sealing part 15. The sealing part 15 is formed after the housing chamber 10a is filled with the liquid, and interrupts the communication between the filling path 12a and the housing chamber 10a. The filling part 12 is formed to cause the liquid to fill the housing chamber 10a from the bottom part 10d side of the housing chamber 10a. The sealing part 15 is formed along the bottom part 10d.

Due to this, in the bag 1, when the liquid is discharged from the housing chamber 10a, the liquid flows into the discharging path 11a along the sealing part 15 and the bottom part 10d of the housing chamber 10a. Thus, in a case of discharging the liquid from the housing chamber 10a, the bag 1 can easily discharge the liquid from the housing chamber 10a to the discharging path 11a. In a case of discharging the liquid from the housing chamber 10a, the bag 1 can prevent the liquid from remaining in the housing chamber 10a. Accordingly, the bag 1 can accurately supply the liquid housed in the housing chamber 10a to a flow channel on a side of the cartridge configured to prepare iPS cells.

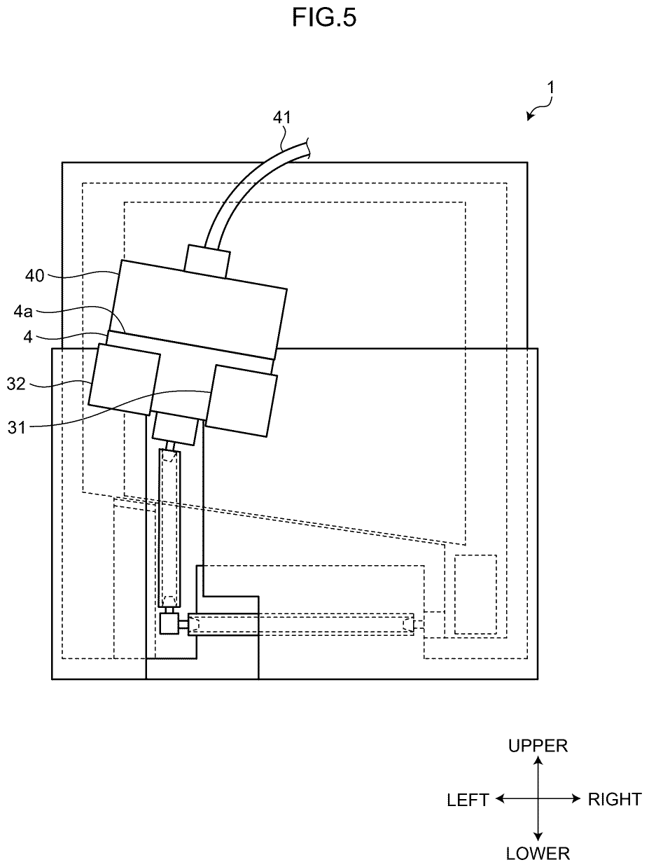

As illustrated in FIG. 5, the second holding parts 31 and 32 may be formed so that the connecting surface 4a of the connector 4 held by the second holding parts 31 and 32 is inclined with respect to the horizontal direction. FIG. 5 is a front view schematically illustrating the bag 1 according to a modification. For example, the second holding parts 31 and 32 are disposed so that the connecting surface 4a of the connector 4 is inclined to face a base part side of the closed-system tube 41 of the cartridge for preparing iPS cells. Due to this, the user can easily connect the connector 4 with the external connector 40. The bag 1 can reduce a load on the closed-system tube 41 when the connector 4 is connected with the external connector 40.

The bag 1 may include any one of the peripheral wall part 10b and the energizing part 13. The bag 1 may include any one of the peripheral wall part 10b and the cylindrical member 14.

The above embodiment has described the case in which the separate unit connected to the bag 1 is the cartridge configured to prepare iPS cells, and the cartridge is installed in an iPS cell preparing device to prepare iPS cells, but the embodiment is not limited thereto. For example, the separate unit connected to the bag 1 may be the iPS cell preparing device, and prepare iPS cells without using the cartridge. The embodiment described above can also be applied to a bag that houses a sample or a reagent used configured to prepare or processing cells and the like other than iPS cells.

According to the above embodiment, a dead volume of a bag can be suppressed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A bag comprising:
a housing part including a housing chamber configured to house a liquid as a sample or a reagent;
a discharging part including a discharging path configured to discharge the liquid from the housing chamber; and
a discharging pipe device connected to the discharging part to discharge the liquid from the housing chamber via the discharging path, wherein
the discharging pipe device is connected to the discharging part to extend from the discharging part toward a direction having a horizontal direction component.

2. The bag according to claim 1, wherein
the discharging part is disposed on a lower side of the housing part,
the discharging path communicates with a lower end of the housing chamber, and
a bottom part of the housing chamber is inclined toward the discharging path.

3. The bag according to claim 1, wherein
a bottom part of the housing chamber is inclined toward a lower side, and
the liquid is guided to the discharging path along the bottom part of the housing chamber when the liquid is discharged from the housing chamber.

4. The bag according to claim 1, wherein
the housing part is formed by joining part of a first sheet with part of a second sheet, and
the housing part includes a peripheral wall part rising from a peripheral part as a joining portion of the first sheet and the second sheet.

5. The bag according to claim 1, comprising an energizing part housed in the housing chamber, wherein
the energizing part energizes a side wall part of the housing part toward an outer side when the liquid is discharged from the housing chamber.

6. The bag according to claim 1, wherein
the discharging part is formed by joining part of a first sheet with part of a second sheet, and
the discharging part includes a peripheral wall part rising from a peripheral part as a joining portion of the first sheet and the second sheet.

7. The bag according to claim 1, comprising a cylindrical member housed in the discharging path.

8. The bag according to claim 1, wherein the discharging part is formed integrally with the housing part.

9. The bag according to claim 1, comprising:
a connector configured to connect the discharging pipe device with an external connector as a separate unit to allow the liquid to flow into the external connector from the discharging pipe device; and
a holder configured to hold the housing part, the discharging part, and the connector.

10. The bag according to claim 9, wherein the holder holds the connector on an upper side of the discharging part.

11. The bag according to claim 9, wherein
the discharging pipe device comprises:
a first tube connected to the discharging part;
a second tube connected to a lower surface of the connector; and
a joint configured to connect the first tube with the second tube.

12. The bag according to claim 1, comprising a filling part including a filling path configured to fill the housing chamber with the liquid and formed to extend from the housing part toward a lower side.

13. The bag according to claim 12, comprising a sealing part configured to be formed after the housing chamber is filled with the liquid to interrupt communication between the filling path and the housing chamber, wherein
the filling part is formed to fill the housing chamber with the liquid from a bottom part side of the housing chamber, and
the sealing part is formed along the bottom part.
